SEQUENCE LISTING (1 sequence)

SEQ ID NO: 1 - DNA, 72 bp, Homo sapiens
  misc_feature: Synthetic anti-MUC1 aptamer
gggagacaagaataaacgctcaagcagttgatcctttggataccctggttcgacaggaggctcacaacaggc

(19) United States
(12) Patent Application Publication
(10) Pub. No.: US 2021/0032626 A1
(43) Pub. Date: Feb. 4, 2021

(54) Material for capturing circulating cells in the blood, method of preparation and use

(71) Applicant: I.CERAM Entreprise, LIMOGES (FR)

(72) Inventors: ANDRE KERISIT, LIMOGES (FR); EVELYNE POLI, COUZEIX (FR); GUISLAINE BARRIERE, SAINT LEONARD DE NOBLAT (FR); GUILLAUME LEVEQUE, NIEUL (FR); MICHEL RIGAUD, FEYTIAT (FR)

(73) Assignee: I.CERAM Entreprise, LIMOGES (FR)

(21) Appl. No.: 16/942,047

(22) Filed: Jul. 29, 2020

(30) Foreign Application Priority Data
Aug. 1, 2019 (FR) 1908831

(51) Int. Cl. C12N 15/115; G01N 33/574; A61M 1/36

(52) U.S. Cl. CPC C12N 15/115; G01N 33/57492; A61M 1/3687; A61M 2202/09; C12N 2310/351; C12N 2320/10; C12N 2310/16

(57) ABSTRACT

A material chosen from alumina ceramic grafted with at least one aptamer, at least one antibody or their combination for capturing circulating foreign cells, preferably circulating tumor cells, the method of preparing such material, its use and the device comprising it are disclosed.

### REFERENCE TO SEQUENCE LISTING SUBMITTED VIA EFS-WEB

This application includes an electronically submitted sequence listing in .txt format. The .txt file contains a sequence listing entitled “6670-0115PUS1_ST25.txt” created on Oct. 21, 2020 and is 629 bytes in size. The sequence listing contained in this .txt file is part of the specification and is hereby incorporated by reference herein in its entirety.

### FIELD OF THE INVENTION

The field of the invention is that of materials allowing the capture of foreign cellular elements circulating in the blood, preferably circulating tumor cells. More precisely, the present invention relates to an alumina ceramic material onto which is grafted at least one aptamer and/or at least one antibody allowing the capture of foreign cellular elements circulating in the blood, preferably circulating tumor cells. The present invention also relates to a method for preparing such a material, a device comprising such a material, as well as the use of the material according to the invention for the capture of foreign cellular elements circulating in the blood, preferably circulating tumor cells.

### TECHNICAL BACKGROUND

Cancer is a disease resulting from the excessive and uncontrolled proliferation of abnormal or malignant cells which may affect any part of the body. When these malignant cells multiply, they form a mass referred to as a “tumor”. Cells sometimes break away from the tumor and pass into the blood and/or lymphatic circulation, spreading throughout the body and forming secondary tumors also known as metastases. These cells are called circulating tumor cells (CTC). It is estimated that 95% to 98% of solid tumors produce CTCs.

Globally, it is also estimated that 70% of breast cancer deaths, as well as 80% of lung cancer deaths, and 40% of prostate cancer deaths, are due to metastasis.

Moreover, the effectiveness of treatment in cancer depends mainly on the possibility of destroying the tumor. Surgery removes the primary tumor. However, the discovery of even a single metastasis means that circulating tumor cells circulate in the blood or lymph, and may nestle in another organ at any time to form another metastasis. In this case, surgery is not sufficient to treat the cancer, and it is necessary to switch to stronger chemotherapeutic treatments. The presence of circulating tumor cells in the blood and/or lymph, therefore, means that there is a risk to the patient of developing metastases in other organs.

Circulating tumor cells are also of interest in terms of prognosis. For example, in the case of breast cancer, it is estimated that the threshold of 5 CTC in a 7.5 ml sample of peripheral blood constitutes a good prognosis for the patient. Beyond this threshold, it is a poor prognosis.

To date, there are many counting technologies but few known methods to capture and eliminate circulating tumor cells. We know, for example, the technique of identification and counting of circulating tumor cells marketed under the name of the CELLSEARCH® System, which is currently the only technique validated by the American Food and Drug Administration (FDA). This technique uses immunomagnetic beads, i.e. magnetic nanoparticles coated with antibodies directed against the proteins EpCam, CD45, cytokeratin 8 and/or cytokeratin 19, making it possible to detect and accurately count CTCs in a 7.5 ml sample. of blood. This technique is limited to the detection and enumeration of CTCs from breast, colon or prostate cancer.

We may also cite the use of beads or nanoparticles on which antibodies are attached to bind circulating tumor cells for their study and characterization. Such beads are, for example, marketed by the company QIAGEN under the names AdnaTest® BreastCancerSelect, or AdnaTest® BreastCancerDetect, or for the enrichment of CTC of breast cancer from a sample of peripheral blood of a patient. The beads have on their surface antibodies specific for the ligands GA733-2, MUC-1 and HER-2.

Other techniques implement the attachment of aptamers for the detection of circulating tumor cells, these aptamers being attached to chips of silica oxide or graphene oxide, or magnetic columns comprising particles of iron oxide.

Due, in particular, to the volume of sample tested, which may not exceed 10 ml, all these techniques are limited to diagnostic purposes or for monitoring the progression of cancer in a patient who has been operated on and/or who has undergone chemotherapy. However, neither of these techniques can capture the circulating tumor cells from a patient's blood for elimination. In fact, these techniques cannot be used in practice to rid a patient's blood of circulating tumor cells, and, in fact, do not have this objective.

However, the capture and elimination of such cells from the body could prevent and/or reduce the risk of tumor development, which would greatly improve the chances of survival of a cancer patient.

Such an objective requires taking into account a number of factors. First, the physiological, chemical and molecular characteristics of circulating tumor cells depend, on the one hand, on the type of primary cancer from which they have emerged and, on the other hand, on the stage of disease progression. In fact, the cancer cells of a newly-formed cancer may have different markers from the cells of a less recent cancer. The system developed must, therefore, be adaptable. Second, the capture of circulating tumor cells from a clinical perspective requires the development of a system allowing the treatment of the total blood volume of a patient, i.e. between 3 and 5 l of blood per patient, or even the treatment of several times this volume as is the case with renal dialysis. Third, the system of capture must be perfectly biocompatible and inert with respect to the blood elements. In particular, the material allowing the capture of circulating tumor cells should not react chemically or physically with the blood elements, preferably should not retain blood elements other than the circulating tumor cells and should not release elements potentially dangerous for the patient into the extracorporeal bloodstream.

In addition to circulating tumor cells, the blood may sometimes be contaminated by the presence of foreign cellular elements, potentially pathogenic, such as viruses, fungi, bacteria. Blood may also include potentially toxic chemicals such as lead, arsenic, pesticides, insecticides, etc.

International application WO 2017/186626 relates to an extracorporeal blood filtration device for capturing circulating tumor cells present in the blood of a patient, as well as a method for capturing circulating tumor cells using such a system. According to this technique, the device comprises one or more chambers as well as a helical screw in the form of an Archimedes screw, the walls of the chambers as well as the screw having on their surface a combination of six antibodies and/or aptamers making it possible to capture circulating tumor cells present in a patient's blood. However, this application does not disclose a concrete example of a material allowing the implementation of this technique.

There is, therefore, a need to create materials making it possible to capture and retain the circulating tumor cells.

### SUMMARY OF THE INVENTION

These objectives are achieved by the material according to the present invention, its use, as well as a method of grafting said material and its use.

The present invention relates to an alumina ceramic material grafted with at least one aptamer, at least one antibody, or combinations thereof.

Advantageously, at least one aptamer, at least one antibody or a combination thereof, is grafted to the alumina ceramic material by a binding molecule.

Advantageously, the binding molecule is a trialkoxysilane of formula N⁻═N⁺═N-L-Si(OR1)(OR2)(OR3) in which L is a linear or branched cyclic, divalent hydrocarbon radical comprising 1 to 20 atoms of carbon, preferably 1 to 10 carbon atoms, and R1, R2, R3 are identical or different and represent a linear or branched alkyl group comprising 1 to 6 carbon atoms, preferably 1 to 3 carbon atoms.

Preferably, the binding molecule is 3-azidopropyltriethoxysilane.

Advantageously, the alumina ceramic material has at least one surface in contact with the blood of a patient, said surface not being porous.

Preferably, the patient is human.

Preferably, the surface of the material in contact with the patient's blood has zero open porosity.

Preferably, the surface of the material in contact with the patient's blood is impermeable.

Advantageously, the alumina ceramic is a dense alumina.

Advantageously, the material is grafted by at least one aptamer.

Advantageously, at least one aptamer is single stranded DNA.

Advantageously, at least one antibody is formed by the whole antibody, the Fv fragment, the Fab fragment or the F(ab′)2 fragment.

Advantageously, the at least one aptamer or at least one antibody is an aptamer substituted by or an antibody substituted by at least one linear or branched cyclic hydrocarbon group comprising a terminal alkyne function having the formula —C≡CH and from 3 to 20 carbon atoms, preferably from 10 to 15 carbon atoms, said hydrocarbon group being optionally substituted by one or more heteroatoms.

Advantageously, the heteroatom (s) may be chosen from among O, S, N, P or a halogen, preferably from among O, N, P, preferably the heteroatoms are oxygen atoms.

Preferably, at least one aptamer is substituted at its 5′ end.

Advantageously, the at least one aptamer or the at least one antibody is capable of binding to at least one of the proteins chosen from mucin-1 (MUC-1), EpCam, HER-2, EGFR, vimentin, E-cadherin, N-cadherin, OB-cadherin, CD44v6, CD44v8, PSA, cytokeratin 8, cytokeratin 19, ABC-G2 and their combinations.

Preferably, the at least one aptamer or the at least one antibody binds to at least the protein mucin-1 (MUC-1).

Advantageously, the at least one aptamer or at least one antibody is linked to at least one labeling molecule.

Preferably, the at least one aptamer is linked to at least one labeling molecule at its 3′ end.

Advantageously, the at least one labeling molecule is a fluorophore.

Advantageously, the fluorophore is chosen from phycoerythrin (PE), fluorescein isothiocyanate (FITC), the Alexa Fluor™ range, green fluorescent protein (or GFP) or combinations thereof.

Preferably, the fluorochrome is fluorescein isothiocyanate (FITC).

An object of the present invention is also a method for preparing the material according to the invention comprising the grafting of the alumina ceramic with at least one aptamer, at least one antibody, or a combination thereof.

Advantageously, the at least one aptamer, at least one antibody, or their combination, is grafted to said alumina ceramic material by a binding molecule, said method comprising at least the following steps:
binding of the binding molecule to the alumina ceramic material by reaction of one or more alkoxy functions of the binding molecule with one or more hydroxyl groups of the alumina ceramic material; and
binding of the aptamer or antibody to the binding molecule by click chemistry reaction.

Advantageously, the at least one aptamer or the at least one antibody is an aptamer substituted by or an antibody substituted by at least one linear or branched cyclic hydrocarbon group comprising a terminal alkyne function having the formula —C≡CH and from 3 to 20 carbon atoms, preferably from 10 to 15 carbon atoms, said hydrocarbon group being optionally substituted by one or more heteroatoms.

Advantageously, the aptamer or the antibody is substituted and the binding of the substituted aptamer or of the substituted antibody to the binding molecule is carried out by reaction of the terminal alkyne function of the substituted aptamer or the substituted antibody with the terminal azide function of formula —N═N⁺═N⁻ of the binding molecule.

Advantageously, the step of binding the binding molecule to the alumina ceramic material is carried out in the presence of an organic solvent.

Preferably, the organic solvent is toluene.

Preferably, the step of binding the binding molecule to the alumina ceramic material is carried out under anhydrous conditions.

Advantageously, the step of binding the binding molecule to the alumina ceramic material is carried out under an inert atmosphere.

Advantageously, the method according to the invention may comprise a step before the step of binding the binding molecule to the alumina ceramic material in which the alumina material is heated beforehand, preferably to a temperature above 100° C.

Advantageously, the step of binding the binding molecule to the alumina ceramic material is carried out at a temperature ranging from 30 to 120° C., preferably from 50 to 100° C.

Advantageously, the step of binding the substituted aptamer or the substituted antibody to the binding molecule is carried out in the presence of one or more organic solvents, preferably acetonitrile or an amine, preferably trimethylamine or triethylamine.

Advantageously, the step of binding the substituted aptamer or the substituted antibody to the binding molecule is carried out in the presence of a catalyst, supported or unsupported.

Advantageously, the catalyst may be a metal catalyst, preferably a copper catalyst.

Preferably, the copper catalyst is copper I bromotris(triphenylphosphine).

Advantageously, the step of binding the substituted aptamer or the substituted antibody to the binding molecule is carried out at a temperature ranging from 30 to 120° C., typically from 50 to 100° C.

Advantageously, the step of binding the binding molecule to the alumina ceramic material is carried out before the step of binding the substituted aptamer or the substituted antibody to the binding molecule.

The present invention also relates to a device for capturing circulating foreign cells comprising the material as described above and at least one housing.

Preferably, the capture device is a circulating tumor cell capture device.

Advantageously, the device is an extracorporeal capture device.

Advantageously, the housing of the device according to the invention has at least one inlet for the entry of blood into the housing and one outlet for the exit of blood from the housing.

Advantageously, the device according to the invention further presents a tool for capturing circulating foreign cells.

Advantageously, the housing has a tubular shape.

Advantageously, the capture tool is in the form of a helical screw or an Archimedean screw comprising an axle of rotation.

Advantageously, the axle of rotation comprises a magnet at each of its ends.

Advantageously, the housing of the device comprises a magnet.

An object of the present invention is also the material according to the invention for its use in capturing circulating foreign cells, preferably circulating tumor cells.

Advantageously, the material according to the invention is used to improve the effectiveness of chemotherapy.

Advantageously, the material according to the invention is used to reduce the risk of metastasis formation.

The object of the present invention is also an aptamer, an antibody or their combination grafted onto an alumina ceramic material according to the invention for the capture of circulating foreign cells, preferably circulating tumor cells.

Advantageously, the circulating tumor cells originate from breast cancer, lung cancer, prostate cancer, colorectal cancer, bladder cancer, stomach cancer, melanoma.

The present invention also relates to a method for capturing circulating foreign cells, preferably CTCs, using the material according to the invention.

### DETAILED DESCRIPTION OF THE INVENTION

The present invention relates to an alumina ceramic material grafted with at least one aptamer, at least one antibody, or a combination thereof. For the purposes of the invention, the grafting of at least one aptamer, at least one antibody or their combination, is covalent. Preferably, the alumina ceramic has a percentage of alumina greater than 95% by weight, more preferably greater than 99% by weight.

In the context of the invention, alumina has the formula Al₂O₃. Alumina ceramic typically has hydroxyl groups on its surface.

The alumina ceramic material according to the invention has the advantage of being biocompatible and chemically inert towards body fluids. Thus, unlike silica, alumina ceramic does not degrade over time and, therefore, will not release any element into the patient's blood. On the other hand, silica cannot be densely shaped like ceramic.

According to one embodiment, the material according to the invention may be grafted with a combination of aptamers, a combination of antibodies, or a combination of aptamers and antibodies.

The material according to the invention has at least one surface in contact with the patient's blood. The at least one aptamer, at least one antibody, or combinations thereof, are grafted onto the surface in contact with the patient's body fluids. Although the present invention finds its application in the treatment of the blood of a patient, it should be understood that it may also be used to treat any other body fluid of a patient. In the context of the present invention, by “body fluid” is meant any fluid produced by the human body such as blood, serum, urine, lymph, chyle etc. Preferably, the patient is human.

In one embodiment, the material according to the invention has a surface in contact with the blood of a patient, said surface not being porous. In the context of the present invention, it is considered that a surface is non-porous when the surface has pores whose size is less than 5 μm, preferably less than 2 μm, more preferably less than 1 μm. In a particularly preferred embodiment, the pore size of the surface of the alumina ceramic material is zero. The open porosity of the surface in contact with the patient's blood is therefore zero. Pore size may be measured by microscopy with image analysis and “diameter” measurements. Porosity may also be measured by mercury porosimetry. The porosity is defined by the ratio between the volume occupied by the pores over the total volume of the ceramic, the total volume comprising the sum of the volume of the pores and the volume of the alumina.

The alumina ceramic material according to the invention is, therefore, biocompatible and hemocompatible: it does not cause any hemolysis or coagulation reaction. In the context of the present invention, it is considered that a material is hemocompatible if it allows the preservation of the osmotic acidobasic equilibria of the blood, if it does not alter or capture plasma proteins, the figured elements of blood, the vascular tissue cells, and if it does not activate the hemostasis and complement systems. In other words, the low porosity of the surface of the material in contact with the patient's blood, and preferably its absence of porosity, allows the material according to the invention to be used for the treatment of the volume of this blood is completely harmless to the patient.

In fact, according to the invention, the alumina ceramic material grafted with at least one aptamer or antibody is intended to come into contact with the patient's blood to capture the circulating foreign cells, preferably the CTCs. If the material according to the invention were porous, blood elements of the patient would be likely to be retained in the pores of the material. Now, an objective of the present invention is to provide a material which captures only circulating foreign cells, preferably CTCs, in the patient's blood and not the elements of the patient's blood such as red blood cells, leukocytes, platelets, chemical elements, without inducing an undesirable reaction such as hemolysis, denaturation of plasma proteins or aggregation of platelets, for example. In other words, the surface of the material according to the invention is impermeable to blood, i.e. it does not allow blood to pass through.

An alumina ceramic which is particularly suitable for the implementation of the invention may be a dense ceramic. In the context of the present invention, it is considered that an alumina ceramic material is dense if its density is greater than 3.5 g/cm³, preferably at least 3.8 g/cm³. Density is measured by dividing the mass of the material by its volume. Preferably, the ceramic material complies with DIN VDE 0335.

The present invention, therefore, provides a material, biocompatible and chemically inert with respect to the patient's body fluids and, in particular, blood. The grafting of the alumina ceramic material according to the invention with at least one aptamer or at least one antibody makes it possible to capture the undesirable elements circulating in the blood of a patient. In the context of the present invention, “capturing” is understood to mean that the foreign cell binds to the aptamer and/or the antibody in a sufficiently strong manner so that it does not detach itself and return to the patient's blood. In particular, when at least one aptamer or antibody is specific for a CTC marker, the material according to the invention makes it possible to capture the CTCs circulating in the patient's blood and to eliminate them. The use of the material according to the invention may, therefore, be used for therapeutic purposes to eliminate CTCs, or, at the very least, to reduce the number of CTCs in the blood of a patient in order to reduce the risk of metastasis formation. The material according to the invention also makes it possible to improve the effectiveness of chemotherapy. In fact, by reducing the number of CTCs in the patient's blood, the material according to the invention allows the chemotherapeutic substances to be more effective without increasing their dosage.

The material according to the invention may also be grafted with several different aptamers or antibodies, or a combination of antibodies and aptamers. Aptamers or antibodies may be either:
directed against different parts of the same target molecule, or
directed against different molecules, or
directed against a combination of these two options, i.e. the material according to the invention may comprise several aptamers or antibodies, some of which are directed against the same target while the other aptamers or antibodies are directed against another target.

Preferably, the aptamer or antibody is custom-made for each patient. In other words, the aptamer or antibody may be designed to react specifically with markers expressed by cells circulating in the patient's blood.

Aptamers and/or antibodies may be distributed uniformly on the surface of the material, or heterogeneously. Preferably, the aptamers or antibodies are distributed homogeneously on the surface of the ceramic so as to effectively capture and remove foreign elements from the blood. The concentration of aptamers and/or antibodies grafted on the surface of the material may be between 0.01 nmol/cm² and 10 nmol/cm², preferably between 0.1 nmol/cm² and 1 nmol/cm², more preferably between 0.3 nmol/cm² and 0.5 nmol/cm².

The material according to the invention may also be grafted with at least one anticoagulant agent to prevent the patient's blood from clotting during processing of the material for the capture and elimination of CTCs. Preferably, the at least one anticoagulant agent may be selected from heparin, an anti-vitamin K agent, a direct factor Xa inhibitor, a direct thrombin inhibitor, or combinations thereof.

The anti-vitamin K agent may be selected from coumarin derivatives, indanedione derivatives or combinations thereof. Coumarin derivatives may be acenocoumarol and warfarin. Indanedione derivatives may be fluindione.

Any anticoagulant agent authorized by the health authorities is suitable for implementing the present invention.

Aptamer

According to a preferred embodiment of the invention, the ceramic material is grafted with one or more aptamers. Preferably, one or more aptamers is covalently grafted to the surface of the material.

In the context of the present invention, an aptamer is a synthetic oligonucleotide making it possible to bind a specific ligand. The term “oligonucleotide” means a DNA or RNA sequence of one hundred base pairs or less. Their selectivity and binding properties are comparable to those of antibodies. The oligonucleotide may be single-stranded ribonucleic acid (RNA) or deoxyribonucleic acid (DNA). Preferably, the aptamer is single stranded DNA.

The aptamer is custom-made to be specific for markers expressed by tumor cells. Those skilled in the art are familiar with the methods of synthesis and selection of aptamers. Among these methods, mention should be made of the SELEX method (Systematic Evolution of Ligands by EXponential enrichment). Briefly, this selection method is an iterative method, based on repeating the steps until the desired aptamer is obtained. It requires a library of oligonucleotides whose 5′ and 3′ ends are known and whose central sequence is random. The randomness of the central sequence is obtained by reacting the four bases of DNA or RNA during the elongation step. The four bases of DNA are adenine, cytosine, guanine and thymine, while the four bases of RNA are adenine, cytosine, guanine and uracil. This makes it possible to obtain oligonucleotides of different length and of different sequence. The next step is to select the oligonucleotides that bind with the best possible affinity to the target molecule. Several techniques known to those skilled in the art allow the separation of oligonucleotides which have recognized the target molecule from other oligonucleotides which do not recognize the target. Examples of a separation method include native gel affinity chromatography, membrane separation, or the use of magnetic beads. The oligonucleotides are then separated from the molecule of interest and are amplified by cloning or by PCR. The steps of selecting the oligonucleotides and of amplification are repeated several times in order to obtain oligonucleotides having the high affinity for the target molecule. The selected oligonucleotides are called aptamers. The SELEX technique provides good results and oligonucleotides with a very low dissociation constant.

The aptamer has selectivity and affinity properties comparable to those of antibodies. The specificity and affinity properties of aptamers allow their use in order to capture the circulating foreign cells, preferably CTCs, in the blood of a patient and to bind them sufficiently strongly so that a circulating cell does not detach from the aptamer to which it is bound and does not return to the patient's blood. Thus, the material according to the invention makes it possible to capture and remove the circulating foreign cells, preferably the CTCs from the blood of a patient.

Antibodies

According to one embodiment, the material according to the invention may be grafted with one or more antibodies. At least one antibody is formed by a whole antibody, an Fv fragment, a Fab fragment or an F(ab′)2 fragment. Preferably, one or more antibodies are covalently grafted to the surface of the material.

The term “antibody”, as used herein, refers to immunoglobulin molecules made up of four polypeptide chains, two heavy chains (H) and two light chains (L) interconnected by disulfide bonds. Each heavy chain is made up of a heavy chain variable region (VH) and a heavy chain constant region. The constant region of the heavy chain is composed of three domains, CH1, CH2 and CH3. Each light chain is composed of a light chain variable region (VL) and a light chain constant region. The constant region of the light chain is composed of a CL domain. The VH and VL regions may be subdivided into regions of hypervariability, called complementarity-setermining regions (CDRs), interspersed with more conserved regions, called framework regions (FR). Each VH and VL is composed of three CDRs and four FRs, arranged in amino-terminus to carboxy-terminus in the following order: FR1, CDR1, CDR1, FR2, CDR2, FR3, CDR3, FR4.

Antibody portions, such as Fv, Fab, and F(ab′)2 fragments, may be prepared from whole antibodies using conventional techniques, such as papain or pepsin digestion of whole antibodies. In addition, the Fv, Fab and F(ab′)2 antibodies or antibody portions may be obtained using conventional recombinant DNA techniques. The term “Fv fragment” is understood to mean the smallest fragment constituted by the heavy and light chains of the variable region and retaining the binding properties of the antibody. The term “Fab fragment” means the monovalent fragment of antibody formed by the entire light chain (variable part and constant part) and part of the heavy chain. By “fragment F(ab′)2” is meant the association of two Fab fragments linked by the hinge of the antibody.

The term “isolated antibody”, as used herein, refers to an antibody which is essentially free from other antibodies having different antigenic specificities (e.g. an isolated antibody which specifically binds MUC-1 is essentially free of antibodies that specifically bind to antigens other than MUC-1). In addition, an isolated antibody may be practically free from other cellular and/or chemical substances.

Antibodies suitable for carrying out the invention may be polyclonal or monoclonal antibodies, produced by any method well known to those skilled in the art. Preferably, the at least one antibody is a monoclonal antibody.

The antibody for implementation of the invention is preferably humanized.

The antibody may further be directed to a specific marker of a foreign cell in the body in order to capture the foreign cells circulating in the blood and to remove these circulating foreign cells.

Preferably, the antibody or the antibody fragment Fv, Fab or F(ab′)2 is grafted to the material according to the invention via a binding molecule.

Substitution

Preferably, the aptamer or the antibody is substituted by at least one linear or branched cyclic hydrocarbon group comprising a terminal alkyne function having the formula C≡CH and from 3 to 20 carbon atoms, preferably from 10 to 15 carbon atoms, said hydrocarbon group possibly comprising at least one heteroatom. The presence of the terminal alkyne function allows the aptamer or antibody to react with the binding molecule in a covalent manner.

The heteroatom(s) may be chosen from O, P, N, S and a halogen, preferably from O, N, P, more preferably the heteroatom(s) are oxygen atoms.

For the purposes of the present invention, a “hydrocarbon group” is a group comprising carbon and hydrogen atoms, linear or branched cyclic, substituted or unsubstituted by one or more heteroatoms. The hydrocarbon group may be saturated or unsaturated.

According to one embodiment, the aptamer or the antibody is substituted with a linear or branched hydrocarbon group comprising a terminal alkyne function and comprising from 3 to 20 carbon atoms, said hydrocarbon chain being substituted by at least one function chosen from among the hydroxyl and carbonyl functions. Preferably, the branch(es) of the hydrocarbon group, when present on the hydrocarbon group, comprise from 1 to 5 carbon atoms, preferably from 1 to 3 carbon atoms, advantageously 1 to 2 carbon atoms.

According to one embodiment, the aptamer or the antibody is substituted with a linear or branched alkyl group comprising a terminal alkyne function and at least one carbonyl function and at least one hydroxyl function, said alkyl group comprising from 10 to 15 carbon atoms. Preferably, the branch(es) of the alkyl group, when present on the alkyl group, comprise from 1 to 5 carbon atoms, preferably from 1 to 3 carbon atoms, advantageously 1 to 2 carbon atoms.

The substituted aptamer or the substituted antibody may be commercially available or synthesized according to methods well known to those skilled in the art.

Thus, the grafting of the aptamer or the antibody to the material is strong enough to prevent its detachment from the material and the aptamer, or the antibody is not found in the peripheral blood of the patient.

Circulating Foreign Cells

The material according to the invention makes it possible to capture any element circulating in the blood. The material according to the invention makes it possible, in particular, to capture cellular elements foreign to the organism and found in the blood.

In particular, the foreign cells may be a virus, bacteria, fungus, circulating tumor cell, animal cell foreign to the body, or combinations thereof.

According to a preferred embodiment, the circulating foreign cells are circulating tumor cells. In fact, tumor cells are often distinguished from “normal” cells of the tissue concerned because they exhibit different cell markers.

Markers

The at least one aptamer or at least one antibody is preferably directed to markers of circulating foreign cells.

In one embodiment, the aptamer or the antibody may be directed against a protein of the capsid of a virus or against a protein, sugar or glycoprotein of the capsid of a virus in order to trap the virus. For example, the aptamer or antibody may be directed against hemagglutinin.

In the context of the present invention, the term “sugar” refers to monosaccharides, disaccharides and polysaccharides. Sugars include, but are not limited to, sucrose, glucose, dextrose and other sugars.

In the context of the present invention, a protein designates a biological macromolecule, formed from one or more polypeptide chains. Each of these chains is made up of the chain of amino acid residues linked together by peptide bonds. In the context of the present invention, an amino acid is a carboxylic acid which also has an amine functional group. Such organic compounds therefore have both a carboxyl group —COOH and an amine group, for example a primary amine —NH2 or a secondary amine —NH—. In the context of the present invention, a glycoprotein is a protein carrying one or more oligoside groups, an oligoside being an oligomer formed from a number of monosaccharides, identical or different, by an alpha or beta glycosidic bond.

The aptamer or antibody may also be directed against a molecule found on the surface of a bacterium. By way of example of a bacterial marker, mention may be made of lipopolysaccharides (LPS) present in the outer membrane of Gram negative bacteria.

Regarding circulating tumor cells (CTC), there are different markers depending on the type of cancer the patient has. Scientific literature provides those skilled in the art with the specific markers that they should look for based on a specific type of cancer. Among the markers to which aptamers can bind, may be mentioned the markers EpCam (EPithelial Cell Adhesion Molecule), HER-2 (Human Epidermal growth factor Receptor 2), EGFR (Epithelial Growth Factor Receptor), the protein mucin-1 (MUC-1), vimentin, E-cadherin, N-cadherin, OB-cadherin, CD44v6, CD44v8, PSA (Prostate Specific Antigen), cytokeratin 8, cytokeratin 19, ABC-G2 and their combinations.

The CD44v6 marker is expressed by colon cancer cells. The HER2 and CD44v8 markers are specifically expressed by breast cancer cells. The EpCam, EGFR and E-cadherin markers are specific for epithelial cells and are found expressed on the surface of numerous cells resulting from carcinomas including non small cell lung cancer for example.

Preferably, the aptamer or antibody binds to the mucin-1 protein. Mucin-1 protein is a glycoprotein encoded by the MUC-1 gene in humans, expressed at the apical pole of many epithelial cells.

This protein is particularly overexpressed by circulating tumor cells originating from breast cancer, lung cancer, prostate cancer, colorectal cancer, bladder cancer, stomach cancer, melanoma.

More specifically, circulating tumor cells express a version of MUC-1 with reduced and uncontrolled glycosylation, which makes it particularly antigenic and differentiates it well from the MUC-1 protein expressed by normal cells. The MUC-1 protein is, therefore, a good discriminating marker for circulating tumor cells.

Preferably, the anti-mucin-1 aptamer is a 72 base single stranded DNA oligonucleotide having the following sequence:

(SEQ ID NO: 1)
5′GGGAGACAAGAATAAACGCTCAAGCAGTTGATCCTTT
GGATACCCTGGTTCGACAGGAGGCTCACAACAGGC3′

The material according to the invention grafted with the aptamer or the antibody binding to MUC-1 therefore makes it possible to capture CTCs expressing this marker and to eliminate a significant number of CTCs from various cancers.

In a particularly advantageous embodiment, the material according to the invention is grafted with several aptamers and/or antibodies making it possible to recognize several different markers and/or different parts of a marker.

Binding Molecule

The at least one aptamer and/or the at least one antibody may be grafted to said material by a binding molecule. The binding molecule covalently binds the aptamer and/or the antibody to the accessible hydroxyl functions present on the surface of the alumina ceramic. The binding molecule also acts as a spacer between the aptamer or the antibody and the biocompatible material. In fact, an aptamer or an antibody is a bulky molecule.

Direct binding of the aptamer or antibody to the material would create steric hindrance which would make the aptamer or antibody little or even unavailable to bind to circulating cellular elements.

According to one embodiment, the aptamer is linked to the binding molecule at its 5′ end.

The binding molecule preferably comprises a reactive function capable of reacting with the substituted aptamer or the substituted antibody by a click chemistry reaction. Thus, the binding molecule preferably comprises a terminal azide function —N═N⁺═N⁻ making it possible to react with the terminal alkyne of the substituted aptamer or of the substituted antibody to form a triazole-type ring.

Preferably, the binding molecule is a trialkoxysilane of formula N⁻═N⁺═N-L-Si(OR1)(OR2)(OR3) in which L denotes a divalent linear or branched cyclic hydrocarbon group comprising 1 to 20 carbon atoms, preferably 1 to 10 carbon atoms, and R1, R2, R3 are identical or different and represent a linear or branched alkyl group comprising 1 to 6 carbon atoms, preferably 1 to 3 carbon atoms.

In the context of the invention, an alkyl group denotes a monovalent saturated hydrocarbon chain.

In the context of the invention, an alkoxy group of formula —(OR) denotes an alkyl group bound to an oxygen atom.

According to one embodiment, L is a divalent, linear or branched hydrocarbon group comprising from 1 to 10 carbon atoms. Preferably, L is a divalent, linear or branched alkyl group comprising from 1 to 10 carbon atoms. More preferably, L is a linear alkyl group comprising from 1 to 6 carbon atoms.

Preferably, the binding molecule is 3-azidopropyltriethoxysilane (AzPTES).

The binding molecule may be commercially available or synthesized according to methods well known to those skilled in the art.

Labeling of Aptamer or Antibody

For the implementation of the present invention, the aptamer or the antibody may also be conjugated to one or more colored or fluorescence label(s). Preferably, the aptamer is labeled at its 3′ end. Labeling of the aptamer or of the antibody may be done by any method well known to those skilled in the art. Labeling of the aptamer may, in particular, be done by a click chemistry reaction between the label and the aptamer.

Preferably, the aptamer or antibody is labeled using a “cold” labeling method. In the context of the present invention, a cold labeling method is a labeling method which does not involve radioactivity. Thus, the material according to the invention is simpler and safer to produce and use. The presence of a marker makes it possible in particular to verify that the grafting of the aptamers has taken place during the production of the material according to the invention.

The aptamer or the antibody may be labeled with one or more fluorophore(s) or with a marker producing a color such as the biotin/streptavidin system, or a combination thereof. By “fluorophore” is meant a chemical compound capable of emitting light after excitation at a given wavelength. Each fluorophore is excitable at a specific wavelength and emits light at a specific wavelength. Those skilled in the art know how to choose the fluorophore(s) suitable for implementing the invention. The fluorophores suitable for the implementation of the present invention may be chosen from the molecules mentioned in Table 1 below:

TABLE 1

| | λ excitation | λ emission | |
|---|---|---|---|
| Name of fluorophore | (nm) | (nm) | CAS No |
| Hydroxycoumarin | 325 | 386 | 43070-85-5 |
| Aminocoumarin | 350 | 445 | 30230-57-0 |
| Methoxycoumarin | 360 | 410 | |
| Cascade ® Blue | (375); 401 | 423 | |
| Pacific ® Blue | 403 | 455 | |
| Pacific Orange ™ | 403 | 551 | 1122414-42-9 |
| Lucifer yellow | 425 | 528 | 82446-52-4 |
| Nitrobenzoxadiazole | 466 | 539 | |
| R-Phycoerythrin (PE) | 480; 565 | 578 | 11016-17-4 |
| PE-Cy5 conjugate | 480; 565; | 670 | |
| | 650 | | |
| PE-Cy7 conjugate | 480; 565; | 767 | |
| | 743 | | |
| Red 613 | 480; 565 | 613 | |
| PerCP (peridinine- | 490 | 675 | |
| chlorophyll-protein | | | |
| complex) | | | |
| True Red | 490, 675 | 695 | 396076-95-2 |
| FluorX ™ | 494 | 520 | |
| Fluorescein | 495 | 519 | 2321-07-5 |
| BODIPY ™ FL (boron- | 503 | 512 | 165599-63-3 |
| dipyrromethene | | | |
| Tetra methyl Rhodamine | 547 | 572 | 107347-53-5 |
| Iso Thio Cyanate (TRITC) | | | |
| X-Rhodamine | 570 | 576 | |
| Lissamine Rhodamine B | 570 | 590 | 3520-42-1 |
| Texas Red (sulfonyl | 589 | 615 | 82354-19-6 |
| chloride) | | | |
| Allophycocyanin (APC) | 650 | 660 | 874103-50-1, |
| | | | 874103-51-2 |
| APC-Cy7 conjugate | 650; 755 | 767 | |
| Cy2 | 489 | 506 | |
| Cy3 | (512); 550 | 570; (615) | |
| Cy3B | 558 | 572; (620) | |
| Cy3.5 | 581 | 594; (640) | |
| Cy5 | (625); 650 | 670 | |
| Cy5.5 | 675 | 694 | |
| Cy7 | 743 | 767 | |
| FITC (fluorescein | 495 | 521 | |
| isothiocyanate) | | | |
| Alexa Fluor 350 | 343 | 442 | 244636-14-4 |
| Alexa Fluor 405 | 401 | 421 | 791637-08-6 |
| Alexa Fluor 430 | 434 | 540 | |
| Alexa Fluor 488 | 499 | 519 | 247144-99-6 |
| Alexa Fluor 500 | 503 | 525 | 798557-08-1 |
| Alexa Fluor 514 | 517 | 542 | 798557-07-0 |
| Alexa Fluor 532 | 530 | 555 | 247145-11-5 |
| Alexa Fluor 546 | 561 | 572 | 247145-23-9 |
| Alexa Fluor 555 | 553 | 568 | 644990-77-2 |
| Alexa Fluor 568 | 579 | 603 | 247145-38-6 |
| Alexa Fluor 594 | 591 | 618 | 247145-86-4 |
| Alexa Fluor 610 | 610 | 629 | 900528-62-3 |
| Alexa Fluor 633 | 632 | 648 | 477780-06-6 |
| Alexa Fluor 647 | 652 | 668 | 400051-23-2 |
| Alexa Fluor 660 | 663 | 691 | 422309-89-5 |
| Alexa Fluor 680 | 680 | 702 | 422309-67-9 |
| Alexa Fluor 700 | 696 | 719 | 697795-05-4 |
| Alexa Fluor 750 | 752 | 776 | 697795-06-5 |
| Alexa Fluor 790 | 782 | 804 | 950891-33-5 |

Preferably, the fluorophore is chosen from among phycoerythrin (PE), fluorescein isothiocyanate (FITC), the Alexa Fluor™ range, green fluorescent protein (GFP). More preferably, the fluorochrome is fluorescein isothiocyanate (FITC).

The labeling of the aptamer or of the antibody may make it possible to verify the good quality of the grafting of the aptamer to the surface of the material. Labeling of the aptamer or antibody may also be used to signal the binding of the aptamer or antibody to the cell marker of the target cell. For example, when the material contains a combination of antibodies and/or aptamers, the antibodies and/or aptamers may be labeled with different colors or fluorophores. The labeling may then be removed by any method well known to those skilled in the art in order to release the aptamer.

Use

Another object of the invention is the use of the material grafted by at least one aptamer and/or at least one antibody as described above for the capture of foreign cells circulating in the blood of a patient, and preferably CTCs. CTCs are preferably derived from the blood of a patient. Preferably, the patient is human.

According to this aspect of the invention, the CTCs are derived from breast cancer, lung cancer, prostate cancer, colorectal cancer, bladder cancer, stomach cancer or melanoma. Preferably, the CTCs originate from a breast cancer, preferably a breast adenocarcinoma. The material according to the invention may also be used to improve the effectiveness of chemotherapy and/or to reduce the risk of formation of metastasis.

In fact, the capture of CTCs from the patient's blood makes it possible to reduce, or even eliminate, the quantity of tumor cells circulating in the patient's blood, and, therefore, to reduce the risks of metastasis. Furthermore, the number of CTCs in a blood sample is a prognostic indicator for the patient: reducing this number of cells per volume of blood sample therefore improves the patient's prognosis. Also, reducing the amount of CTC in the blood allows chemotherapy drugs to be more effective without increasing the dose of the drug to be injected into the patient.

Process for Preparing a Material According to the Invention

An object of the present invention is also a process for preparing the alumina ceramic material according to the invention by grafting at least one aptamer, at least one antibody, or their combination on the alumina ceramic material.

According to one embodiment, the method comprises at least the following steps:
binding of the binding molecule to the alumina ceramic material by reaction of one or more alkoxy functions of the binding molecule with one or more hydroxyl groups of the alumina ceramic material; and
binding of the substituted aptamer or substituted antibody to the binding molecule by click chemistry reaction.

Thus, the material according to the invention is first grafted by the binding molecule, then the at least one substituted aptamer or the at least one substituted antibody is linked to the binding molecule. The step of binding the binding molecule to the alumina ceramic material is therefore carried out before the step of binding the substituted aptamer or the substituted antibody to the binding molecule.

This makes it possible, in particular, to produce the material under mild conditions and to obtain good grafting yields of at least one aptamer and/or antibody on the material. Furthermore, this variant avoids any risk of direct and undesirable bonding of the aptamer or of the antibody to the ceramic material. As explained above, it is desirable that the aptamer or the antibody is bound to the ceramic material via the binding molecule to avoid too rapid saturation of the material by steric hindrance linked to foreign cells, preferably circulating tumor cells, fixed on the aptamers.

In the context of the present invention, the mild condition corresponds to synthetic conditions compatible with the use of a biomolecule, for example using a suitable solvent such as acetonitrile to keep it within a reaction time between 4 h and 8 h, preferably 6 h, and at a low temperature between 30° C.-120° C., preferably about 90° C.

In the context of the present invention, the expression “click chemistry” designates a class of “biocompatible” chemical reactions used to attach a specific biomolecule to a chosen substrate (see https://fr.wikipedia.org/wiki/Chimie_click). Typically, click reactions occur in one-pot synthesis, i.e. a chemical synthesis in which a reagent undergoes several successive and/or simultaneous reactions in a single reaction mixture.

According to one embodiment, the at least one aptamer and/or the at least one antibody is substituted and the grafting of the at least one aptamer and/or the at least one antibody to the ceramic material alumina is made via a binding molecule.

In one embodiment of the invention, the binding of the at least one substituted aptamer or the at least one substituted antibody to the binding molecule by reaction of the terminal alkyne function of the substituted aptamer with the terminal azide function of formula —N═N⁺═N⁻ of the binding molecule. This bond may be effected by an alkyne-azide cycloaddition also called a 1,3-dipolar Huisgen cycloaddition. Typically, the reaction of the terminal alkyne function of the substituted aptamer or of the substituted antibody with the terminal azide function of formula —N═N⁺═N⁻ of the binding molecule makes it possible to form a 1,2,3-triazole type cycle.

According to one embodiment, the step of binding the binding molecule to the alumina ceramic material is carried out in the presence of an organic solvent, such as toluene, preferably under anhydrous conditions, and advantageously under inert atmosphere, typically under argon. Before this bonding step, the alumina material may be heated beforehand, for example to a temperature above 100° C., in order to dry it and remove moisture.

According to one embodiment, the step of binding the binding molecule to the alumina ceramic material is carried out at a temperature ranging from 30 to 120° C., typically from 50 to 100° C.

According to one embodiment, the step of binding the substituted aptamer or the substituted antibody to the binding molecule is carried out in the presence of one or more organic solvents, such as acetonitrile or an amine, for example of the trimethylamine or triethylamine type.

According to one embodiment, the step of binding the substituted aptamer or the substituted antibody to the binding molecule is carried out in the presence of a catalyst, supported or unsupported, for example a metallic catalyst, for example copper, having ligands of the triphenylphosphine type. Among the catalysts which may be used, mention may be made of copper bromotris (triphenylphosphine).

According to one embodiment, the step of binding the substituted aptamer or the substituted antibody to the binding molecule is carried out at a temperature ranging from to 120° C., typically from 50 to 100° C., preferably about 90° C.

The present invention also relates to an aptamer, an antibody or a combination thereof, grafted onto an alumina ceramic material according to the invention for the capture of circulating foreign cells, preferably circulating tumor cells.

The object of the present invention is also a method for capturing circulating foreign cells, preferably circulating tumor cells, using the material according to the invention.

Device

An object of the present invention is also a device for capturing circulating foreign cells, preferably circulating tumor cells, using the material according to the invention as described above.

According to one embodiment, the device according to the invention comprises the material according to the invention as described above as material for capturing circulating tumor cells. The device may, in particular, be similar in its operation to that described in international application WO 2017/186626 A1.

Preferably, the device according to the invention is an extracorporeal device, making it possible to bring the patient's peripheral blood into contact with the grafted material by at least one aptamer according to the invention.

The device according to the invention may comprise a housing making it possible to bring the patient's extracorporeal blood circulation with the material according to the invention, comprising at least one inlet for the entry of blood into the housing and one outlet for the exit of blood from the housing. The housing may house a capture tool.

In one embodiment of the invention, the housing may have a tubular shape.

The capture tool may have a helical shape. In particular, the helical screw or Archimedes screw extends longitudinally about an axle of rotation and is fixed to the capture chamber by means of a fixing system. Preferably, each end of the axle of rotation is provided with a magnet. In this variant, the housing further comprises a magnet, and, in particular, with a pole opposite to that of the magnet present on the end of the axle of rotation. Thus, the presence of the magnets, on the one hand, on the housing and, on the other hand, on the ends of the axle of rotation of the capture tool makes it possible to create a magnetic field which keeps the capture tool in suspension in its housing. Thus by being held in suspension in the housing of the device, the capture tool is not blocked by blood pressure and continues to process the patient's blood.

More precisely, the mobile capture element has the shape of a helix or a spiral. This particular form is used to ensure a regular circulation of the blood fluid and, at the same time, a larger surface in contact with the circulating blood in order to increase the probability of connection between the entity to be removed, for example the tumor cell, and the binding agents, for example the antibody or aptamer. Preferably, the helical surface may extend the entire length of the capture chamber. However, a configuration in which the length of the helix is less than the length of the capture chamber and inside which there are a plurality of helical structures placed in series may also be used. Of course, thanks to such a configuration, the length of the entire device must necessarily be increased.

According to the embodiments of the present invention, the movable capture element is designed to facilitate contact between the reagent surface of the apparatus and the target cells. Thus, any type of movable helix, double helix, or other movable 3D structure designed to facilitate this contact may be included in this invention.

In one embodiment, the capture tool may be manufactured or covered in whole or in part by the material according to the invention. The internal walls of the housing may also be covered in whole or in part by the material according to the invention.

The device according to the invention makes it possible, in particular, to bring the total volume of blood of a patient into contact with the material according to the invention. Depending on the patient's height and weight, it is estimated that a patient's total blood volume is between 3 L and 5 L. Preferably, the device according to the invention makes it possible to bring the total volume of a patient's blood into contact on several occasions, so as to maximize the capture of the patient's circulating tumor cells.

The device according to the invention may also include a pump in order to pump the patient's blood. The device according to the invention may also include filters.

### FIGURES

FIG. 1 is a diagram of the formula of the anti-MUC-1 aptamer substituted with a hydrocarbon group comprising a terminal alkyne.

FIG. 2 is an infrared spectrum of AzPTES as produced prior to its binding to the aptamer and ceramic.

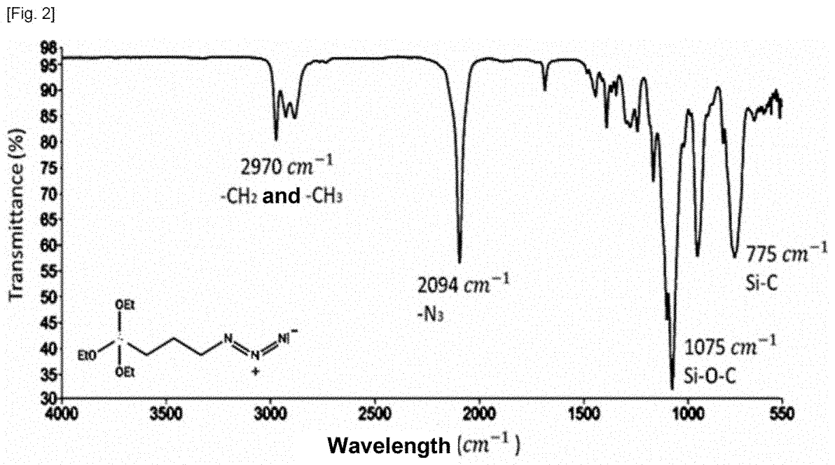

FIG. 3 is a diagram summarizing the reaction on dense alumina according to Example 1.

FIG. 4 shows photographs taken by confocal microscopy of a material according to the invention not grafted (A), of a material according to the invention grafted by an aptamer labeled with FITC according to the invention (B), and a three-dimensional map of the material according to the invention grafted with an aptamer labeled with FITC according to the invention (C).

FIG. 5 shows confocal microscopic photographs of MDA MB231 cells in the presence or absence of an FITC-labeled aptamer.

### EXAMPLES

The following examples are given by way of illustration of the present invention, and are in no way intended to limit its scope.

Described below are examples of preparation by grafting an aptamer onto an alumina ceramic in order to prepare an alumina ceramic grafted with at least one aptamer according to the invention.

The ceramic used is a dense Al₂O₃ alumina ceramic of DIN C799 type, produced by the company SCERAM. According to the supplier's specifications, C799 alumina comprises 99.7% Al₂O₃ alumina and has a density of 3.8 and an absolute density of 3.9 g/cm³. The open porosity of this ceramic is 0%.

The aptamer used is a 72 base single stranded DNA oligonucleotide designed by the SELEX method to bind to the MUC-1 protein and has the sequence:

(SEQ ID NO: 1)
5′GGGAGACAAGAATAAACGCTCAAGCAGTTGATCCTT
TGGATACCCTGGTTCGACAGGAGGCTCACAACAGGC3′

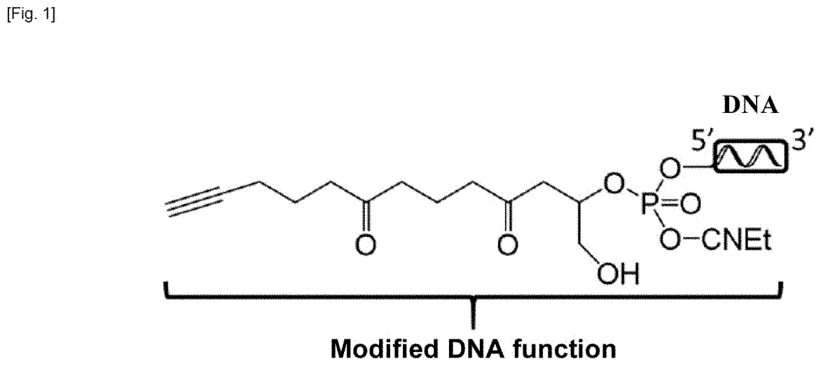

In order to be able to react with other molecules, the aptamer was modified at its 5′ end by the addition of a hydrocarbon group including a terminal alkyne group. The reaction of the azide of the binding molecule with the terminal alkyne group of the substituted aptamer forms a triazole ring. The diagram of the formula of the substituted aptamer is represented in FIG. 1. The aptamer thus substituted is used for the implementation of Examples 1 and 2. The substituted anti-MUC-1 aptamer is produced to order and obtained. commercially.

### Example 1 Method of Grafting an Anti-MUC-1 Aptamer onto a Dense Alumina Ceramic

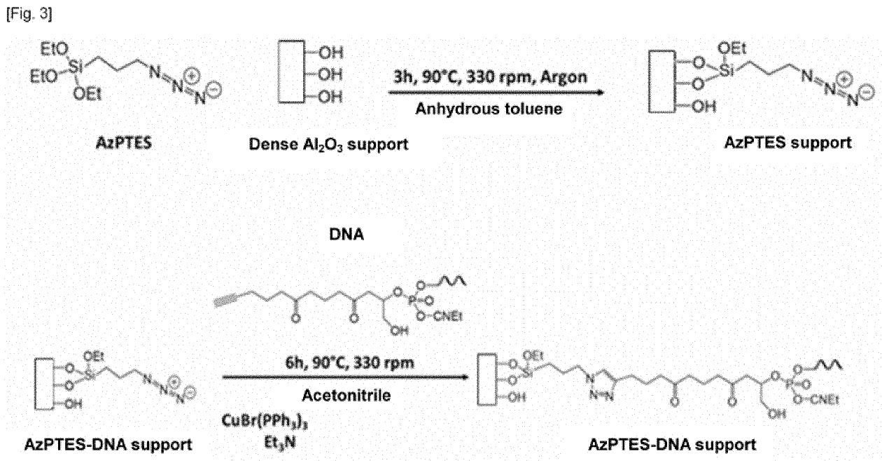

Example 1 illustrates the preparation method according to the invention in which the binding molecule is first bound to the alumina ceramic before a substituted anti-MUC-1 aptamer is bound to the binding molecule. All of the synthetic reactions are summarized in FIG. 3.

### Step 1—Synthesis of the 3-azidopropyl) triethoxysilane Binding Molecule (AzPTES)

1.93 g of chlorosilane (8 mmol), 0.2 g of KI (1.2 mmol) and 1.56 g of NaN₃ (24 mmol) are mixed in a 50 ml Schlenk tube. The system is placed under vacuum and then under an argon atmosphere and 10 ml of DMF (N,N-dimethylformamide) are introduced under argon. The reaction is started for 24 h at 80° C. and 330 rpm. The solution is then yellow. After 24 hours, the solution has turned white and has two phases. Once the medium has returned to ambient temperature, the solution is filtered through a Celite® 545 filter (approximately 1 cm) soaked in 15 ml of DMF on a No. 3 frit, then rinsed with 5 ml of DMF. The DMF is evaporated using a rotary evaporator (70° C., 90 rpm).

Dichloromethane (30 ml) is added to the solution. This is washed twice with 30 ml of water, then the organic phase is dried over MgSO₄, then filtered and placed in a rotary evaporator (40° C., 90 rpm) in order to remove the dichloromethane and any traces of DMF. White residues are observed. The solution is centrifuged for 5 min at 1500 rpm. The oily-looking yellow supernatant containing AzPTES is recovered and then weighed: 1.1 g (4 mmol) of product are recovered. The molar yield of this step is approximately 56%. The AzPTES has the following formula:

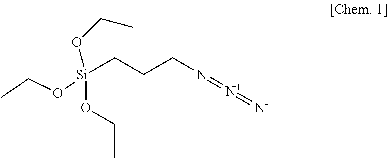 [SMILES: CCO[Si](CCCN=[N+]=[N-])(OCC)OCC]

The infrared and NMR characterization results of AzPTES are as follows:

FR-IR (ATR, resolution 4 cm⁻¹, 8 analyzes/sample): Band observed at 2100 cm⁻¹ characteristic of the N3 group. Other bands observed: 1075 cm⁻¹ characteristic of the Si—O—C group, 775 cm⁻¹ characteristic of the Si—C group. The infrared spectrum of AzPTES is shown in FIG. 3.

1H NMR (500 MHz, CDCl3, 25° C., δ, ppm): 0.69 (m, 2H, CH2-Si), 1.23 (t, 9H, CH3, J=7.0 Hz), 1.71 (m, 2H, CH2), 3.26 (t, 2H, CH2-N3, J=6.9 Hz), 3.83 (q, 6H, CH2-O, J=7.0 Hz)

13C NMR (500 MHz, CDCl3, 25° C., δ, ppm): 7.7 (CH2-Si), 18.3 (CH3), 22.7 (CH2), 53.9 (CH2-N3), 58.5 (CH2-O)

### Step 2—Binding of AzPTES to the Alumina Ceramic

An alumina ceramic pellet 14 mm in diameter and 4 mm thick is used.

The Al₂O₃ alumina ceramic pellet is placed in the oven at 120° C. overnight. A 50 ml two-necked flask is placed in the oven at 120° C. for 5 minutes in order to remove any trace of moisture in the flask. The ceramic is then introduced into the flask. This is placed under vacuum and then under argon. 7 ml of anhydrous toluene is introduced under argon. The mixture is brought to 60° C., 330 rpm. Once the temperature of 60° C. is reached, 2 nmol of AzPTES is introduced in argon, drop by drop, then a refrigerant is installed on the flask and placed under argon. The reaction is carried out for 3 h at 90° C., 330 rpm.

After 3 h, the ceramic is washed with 10 ml of toluene for 2 min in an ultrasonic bath (water, power: 100%, 80 Hz) three times, then once with 10 ml of 95% ethanol.

X-ray photoelectron spectrometry (XPS) analysis detects the presence of silicon and nitrogen, evidence that the binding of DNA-bound AzPTES to alumina ceramic has functioned. In fact, silicon and nitrogen are not part of the chemical elements present in ceramic. The presence of silicon on the surface of the material indicates that the binding of AzPTES to the material has worked and the presence of nitrogen indicates the presence of the single stranded DNA forming the aptamer.

### Step 3—Binding of the Anti-MUC-1 Aptamer to AzPTES Bound to the Alumina Ceramic

4.8 mg of catalyst (CuBr(PPh₃)₃, 1.5 μl of trimethylamine and 7 ml of acetonitrile are mixed with the ceramic bound to AzPTES. The whole is brought to 60° C., 330 rpm. Once the temperature of 60° C. has been reached, the DNA is introduced dropwise. A condenser is placed on the flask and the reaction is carried out for 6 h at 90° C., 330 rpm for binding of the aptamer to AzPTES. The reaction medium is then transparent.

At the end of the reaction, the catalyst (CuBr(PPh₃)₃) is removed by filtration of the solution using a filter (porosity 0.45 μm) on a syringe. The ceramic is washed three times with acetonitrile and then once with ethanol in an ultrasonic bath as described in step 1.

The alumina ceramic is then cleaned with 3 ml of sterile 95% ethanol. The pellet is then stored at 4° C. to avoid or reduce the degradation of the aptamer grafted onto the ceramic. The material thus grafted may be stored for 1 month at 4° C.

The reaction medium and the washing media (acetonitrile and ethanol) are added and then placed in a rotary evaporator at 40° C., 90 rpm. After the solvents have evaporated, 200 μl of ultrapure water are added to the DNA desorbed by the washings.

The ungrafted DNA is then quantified in visible UV at 260 nm and 280 nm according to any method well known to those skilled in the art.

An infrared analysis (ATR, resolution 4 cm⁻¹, 8 analyzes/sample) makes it possible to observe a band at 3325.35 cm⁻¹, characteristic of the —NH functions of DNA, as well as a broad band. at 2114.90 cm⁻¹ characteristic of the alkyne group, and a band at 1639.12 cm⁻¹ characteristic of DNA ketones.

The bond yield of the aptamer on the ceramic material according to the method of Example 2 is about 44%.

### Example 2

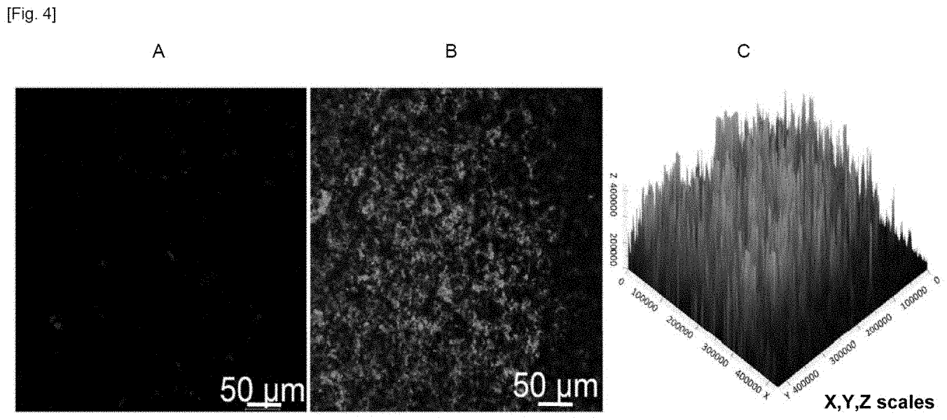

Example 1 was reiterated using an aptamer marked at its 3′ end by the FITC. The material thus prepared was observed under a confocal microscope. An unlabeled alumina ceramic material was also observed as a control: as may be seen in FIG. 4A, no fluorescence is visible on the surface of the material.

It was observed that the material prepared according to the process according to the invention was grafted homogeneously with the aptamer (FIG. 4B). A three-dimensional map was produced by scanning the grafted material of FIG. 4B with a laser over a thickness of 8 μm with a pitch of 0.8 μm, which corresponds to taking a confocal image every 0.8 μm. The digital compilation of these images provides the three-dimensional map shown in FIG. 4C. As this map shows, the material according to the invention is grafted with the aptamer labeled with FITC in a dense and homogeneous manner (FIG. 4C).

### Example 3

In vitro binding assays were performed on MDA MB231 breast cancer cells. MDA MB231 cells are epithelial cells derived from adenocarcinoma of the breast. MDA MB231 cells are cultured according to the supplier's instructions. The cells are then trypsinized, washed and centrifuged for 10 min at 1500 rpm. For the test, 20,000 cells are taken up in 500 μl of cell culture medium without fetal calf serum (FCS). Three conditions are tested:
Negative control: culture medium (20 μl),
FITC alone diluted in the culture medium (20 μl), or
FITC-labeled aptamer in culture medium (20 μl).
   The aptamer used is an aptamer specific for the MUC1 receptor and comprises 72 bases as follows:

(SEQ ID NO: 1)
5′-GGGAGACAAGAATAAACGCTCAAGCAGTTGATCCTT
TGGATACCCTGGTTCGACAGGAGGCTCACAACAGGC-3′

The aptamer is labeled with fluorescein isothiocyanate FITC at its 3′ end according to any technique well known to those skilled in the art.

The cells are then incubated for 1 hour at 37° C. and 5% CO₂ before being transferred to a 24-well plate and incubated 12 h-16 h at 37° C. and 5% CO₂. The cells are then washed and fixed with 500 μl of PBS PAF at 4% before being observed by confocal microscopy, with or without fluorescence, with a magnification ×10.

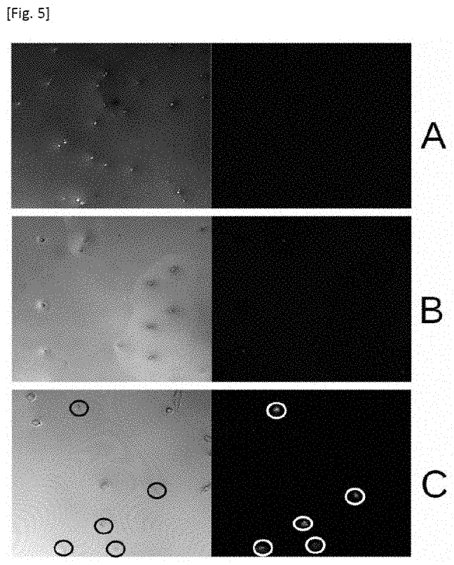

FIG. 5A shows photographs of cells grown in wells under the negative control condition, i.e. cells grown in culture medium without fluorescence marker. The photograph on the left corresponds to the observation without fluorescence while the photograph on the right shows the cells observed with fluorescence. No fluorescence is observed on the cells. The cells, therefore, do not emit spontaneous fluorescence.

FIG. 5B shows photographs of cells grown in wells under the FITC only control condition, i.e. cells grown in culture medium containing FITC but no aptamer. The photograph on the left corresponds to the observation without fluorescence while the photograph on the right shows the cells observed with fluorescence. No fluorescence is observed on the cells. Fluorochrome FITC therefore does not spontaneously bind to cells.

FIG. 5C shows photographs of cells grown in wells with the FITC-labeled aptamer. The photograph on the left corresponds to the observation without fluorescence while the photograph on the right shows the cells observed with fluorescence. As may be seen, the cells on which the FITC-labeled aptamer binds emit fluorescence.

### CONCLUSION

The present invention therefore provides an alumina ceramic material grafted with an aptamer, an antibody or a combination thereof, the grafting being carried out covalently, preferably by means of a binding molecule, as well as a method of preparation of such a material. The material according to the invention therefore makes it possible to provide a biocompatible and hemocompatible material for the capture of foreign cells, preferably circulating tumor cells present in the blood of a patient. The material according to the invention is inert with respect to the patient's blood so that it does not capture any element circulating in the patient's blood other than the targeted foreign cells and that it does not cause any depletion, denaturation or coagulation. or reaction that could harm the patient. The material according to the invention also makes it possible to treat the total blood volume of a patient, or even to allow several passages of the total blood volume in contact with the material to capture and remove the maximum possible number of circulating cells from the patient's blood. The material according to the invention may therefore be used for therapeutic purposes to reduce the risk of metastasis or the risk of cancer recurring. The material according to the invention may also be used to improve the effectiveness of a chemotherapeutic treatment.

The material according to the invention may also be used as part of a device for capturing circulating foreign cells, preferably an extracorporeal device for capturing circulating tumor cells. In fact, unlike the devices of the prior art in which the aptamers and/or antibodies are adsorbed to the surface of the material, the material according to the invention is covalently grafted by at least one aptamer and/or at least an antibody.

This grafting prevents an aptamer and/or an antibody from being detached, carried along by the flow, and from passing into the patient's bloodstream.

1. An alumina ceramic material grafted with at least one aptamer, at least one antibody or combinations thereof.

2. The material according to claim 1 in which at least one aptamer, at least one antibody or a combination thereof is grafted to said alumina ceramic material by a binding molecule.

3. The material according to claim 2 wherein the binding molecule is a trialkoxysilane of formula ⁻N═N⁺═N-L-Si(OR1)(OR2)(OR3) in which L is a linear, branched or cyclic divalent hydrocarbon radical comprising 1 to 20 carbon atoms, preferably 1 to 10 carbon atoms, and R1, R2, R3 are the same or different and represent a linear or branched alkyl group comprising 1 to 6 carbon atoms, preferably 1 to 3 carbon atoms.

4. The material according to claim 1 in which at least one aptamer is single stranded DNA.

5. The material according to claim 1 wherein the at least one aptamer or at least one antibody is an aptamer substituted by or an antibody substituted by at least one linear or branched cyclic hydrocarbon group comprising a terminal alkyne function having the formula C≡CH and from 3 to 20 carbon atoms, preferably from 10 to 15 carbon atoms, said hydrocarbon group being optionally substituted by one or more heteroatoms.

6. The material according to claim 1 having at least one surface in contact with the blood of a patient, said surface not being porous.

7. The material according to claim 1, in which the at least one aptamer or the at least one antibody is capable of binding to at least one of the proteins chosen from mucin-1 (MUC-1), EpCam, HER-2, EGFR, vimentin, E-cadherin, N-cadherin, OB-cadherin, CD44v6, CD44v8, PSA, cytokeratin 8, cytokeratin 19, ABC-G2 and their combinations, preferably the protein mucin-1 (MUC-1).

8. The material according to claim 1 wherein the at least one aptamer, at least one antibody or their combination is linked to at least one labeling molecule.

9. An aptamer, antibody or a combination thereof grafted onto the alumina ceramic material according to claim 1 for the capture of circulating foreign cells, preferably of circulating tumor cells.

10. The aptamer, antibody or a combination thereof for its use according to claim 9 wherein the circulating tumor cells are from breast cancer, lung cancer, prostate cancer, colorectal cancer, bladder cancer, stomach cancer, melanoma.

11. A method for preparing the material according to claim 1, comprising grafting the alumina ceramic with at least one aptamer, at least one antibody or a combination thereof.

12. The method of claim 11, wherein at least one aptamer, at least one antibody or a combination thereof is grafted to said alumina ceramic material by a binding molecule, said method comprising at least the following steps:
binding of the binding molecule to the alumina ceramic material by reaction of one or more alkoxy functions of the binding molecule with one or more hydroxyl groups of the alumina ceramic material; and
binding of the aptamer or antibody to the binding molecule by click chemistry reaction.

13. The method of claim 11 wherein the at least one aptamer or the at least one antibody is an aptamer substituted by or an antibody substituted by at least one linear or branched cyclic hydrocarbon group comprising a terminal alkyne function having the formula C≡CH and from 3 to 20 carbon atoms, preferably from 10 to 15 carbon atoms, said hydrocarbon group being optionally substituted by one or more heteroatoms.

14. The method according to claim 10 wherein the aptamer or the antibody is substituted and the binding of the substituted aptamer or of the substituted antibody to the binding molecule is carried out by reaction of the terminal alkyne function of the aptamer substituted or the antibody substituted with the terminal azide function of formula —N═N⁺═N⁻ of the binding molecule.

15. A device for capturing circulating foreign cells, preferably circulating tumor cells, comprising the material according to claim 1 and at least one housing.

16. The material according to claim 2 in which at least one aptamer is single stranded DNA.

17. The material according to claim 3 in which at least one aptamer is single stranded DNA.

18. The material according to claim 2 wherein the at least one aptamer or at least one antibody is an aptamer substituted by or an antibody substituted by at least one linear or branched cyclic hydrocarbon group comprising a terminal alkyne function having the formula C≡CH and from 3 to 20 carbon atoms, preferably from 10 to 15 carbon atoms, said hydrocarbon group being optionally substituted by one or more heteroatoms.

19. The material according to claim 3 wherein the at least one aptamer or at least one antibody is an aptamer substituted by or an antibody substituted by at least one linear or branched cyclic hydrocarbon group comprising a terminal alkyne function having the formula C≡CH and from 3 to 20 carbon atoms, preferably from 10 to 15 carbon atoms, said hydrocarbon group being optionally substituted by one or more heteroatoms.

20. The material according to claim 4 wherein the at least one aptamer or at least one antibody is an aptamer substituted by or an antibody substituted by at least one linear or branched cyclic hydrocarbon group comprising a terminal alkyne function having the formula C≡CH and from 3 to 20 carbon atoms, preferably from 10 to 15 carbon atoms, said hydrocarbon group being optionally substituted by one or more heteroatoms.
